(19) United States
(12) Patent Application Publication
(10) Pub. No.: US 2012/0109005 A1
(43) Pub. Date: May 3, 2012

(54) APPARATUS AND METHOD FOR VOICE BASED DIAGNOSTIC SUPPORT

(75) Inventors: Siegbert Warkentin, Limhamn (SE); Catarina Erikson, Limhamn (SE)

(73) Assignee: PHIBIOSCIENCE AB, Limhamn (SE)

(21) Appl. No.: 13/319,065

(22) PCT Filed: May 2, 2010
(86) PCT No.: PCT/SE10/50480

§ 371 Date: Jan. 18, 2012

Related U.S. Application Data
(60) Provisional application No. 61/215,772, filed on May 11, 2009.

(30) Foreign Application Priority Data
May 4, 2009 (SE) 0950299-8

(51) Int. Cl. A61B 5/00; A61B 3/032

(52) U.S. Cl. 600/558; 600/559

(57) ABSTRACT

An apparatus (4) is disclosed adapted to diagnose disorders related to the brain of a subject. The apparatus comprises a display (42) for presenting stimuli to said subject, a microphone (46) for registering a sound signal, and a control unit adapted to measure a pause time until articulation of the subject starts in a said sound signal for naming presented stimuli as quickly as possible and in an order as previously presented. Components of said pause time are analyzed for diagnostic support of neuropsychiatric disorders, in particular neurodevelopmental disorders, or brain damage induced disorders. An adjustment of medication dosage or medication type to individual patient needs may be made based on the measurement.

### FIELD OF THE INVENTION

This invention pertains in general to the field of diagnosing health disorder states of a human using a voice signal. More particularly the invention relates to an apparatus, a method, and a computer program for support of diagnosing disorders related to the brain, based on a voice signal.

### BACKGROUND OF THE INVENTION

Disorders related to the brain include neuropsychiatric disorders. One such neuropsychiatric disorder is Attention deficit hyperactivity disorder (ADHD). In more detail, ADHD is a neurodevelopmental behavioural disorder. The worldwide prevalence of this disorder has been estimated to 5.3% in children and adolescents younger than 18 years, and the condition is more common in boys than in girls. The cardinal features are inattentiveness, hyperactivity, and impulsivity. Due to variations in the predominant cardinal features, the disorder is regarded as a heterogeneous and can be subdivided into an Inattentive type (difficulty to focus or paying attention), Hyperactive-Impulsive type (very active and acts without thinking), and a Combined type (inattentive, impulsive, and too active). Although research indicates a strong heritability (about 75%), the disorder does not follow a traditional model of “genetic disease” and is therefore viewed as a complex interaction between genetic, environmental, and dietary factors. The management of ADHD usually involves a combination of family counseling, adjustment of school situation, learning strategies, and often also stimulant medications to reduce the degree of hyperactivity and impulsivity.

In general, cognitive research suggests that children with ADHD have weaknesses in their executive functions (i.e. planning, sustaining attention, ignoring irrelevant information, working memory) and speed of information processing (they process information more slowly than their peers). These weaknesses are often interrelated and may individually or in combination contribute to the child's difficulties in literacy, numeracy, and academic success.

For example, children with either the Inattentive or Combined subtypes of ADHD may have difficulties maintaining information in working memory long enough to be able to perform necessary mental computations to arrive at an answer. In addition to possible limitations in their working memory span, children and adolescents with the disorder are often slower in their ability to process information (information processing speed). Some possible reasons for this may pertain to some of the reported pathophysiology(ies) of the disorder. Although findings are inconsistent, studies have shown a delay in the development of certain brain structures, (i.e. frontal, temporal and parietal cortices) while other structures like the motor cortex mature faster. Studies have also shown a developmental delay of the maturation of brain white matter. Myelinization of axonal processes is a relatively slow and delicate process which continues into mid-life. A developmental delay of white matter tracts will affect the speed by which the brain can process information and will also affect the speed of interaction between various cortical and subcortical areas connected by these tracts. Follow-up studies have provided evidence for a developmental delay starting very early in life. For example, studies of brain morphology (with Magnetic Resonance Imaging) have shown a smaller brain volume of young children with ADHD. Although the brains of children with and without ADHD develop in parallel and the same pace, this difference in brain volume remains into adolescence and probably into also adulthood.

Besides these pathophysiological and morphological findings in ADHD, environmental agents like heavy metals, food additives and sugar have also been implicated in the aetiology of this condition.

Stimulant medication is one of the widely used treatments for ADHD and is often used to alleviate ADHD-symptoms, such as hyperactivity and impulsivity.

Although the use of such medications is increasingly debated, they have shown positive effects, e.g. on cognitive functioning, including processing speed.

There is a need to test, facilitate diagnosis, or provide diagnosis of ADHD. A convenient and non-invasive way is to analyze speech of the patient.

Tannock et al (2000) reported the first investigation that stimulant medication effects naming speed. They found that children with ADHD were impaired in colour naming speed irrespective of comorbid reading disability, while methylphenidate improved the naming speed in these children. Other conclusive evidence has shown that the often reported higher response variability (naming speed irrespective of stimulus) in ADHD is reduced by methylphenidate treatment.

U.S. Pat. No. 6,350,128 ('128) describes a rapid automatized naming method and apparatus. In '128 a method is disclosed for analyzing reading and attentional skills comprised of recording and digitizing sound waves generated by a subject's voice in response to a series of stimuli. Each sound wave is decomposed into a sequence of accumulated articulation and pause periods, and this information is used in relation to reading and attentional skills of children. However, neuropsychiatric disorders are not mentioned in '128. Moreover, phonological output (articulation time) is a less sensitive measure of higher-order cognitive processes performed by the brain. Also, the method and apparatus of '128 is cultural- and educational dependent.

In EP 1639943 a method is disclosed to measure the cognitive capacity to reconstruct from incomplete data original data explainable by correspondence to brain function.

In United States published patent application 2004/0058306 ('306) a verbally based test, system and method is disclosed for testing parietal lobe function, and in particular for Alzheimer's disease. '306 makes use of first order colour stimuli, and first order shapes, letters, numbers, animals and objects, and random combinations of coloured shapes, letters, numbers, animals, and objects. In the main phase of the test administration, three trials are administered to determine the level of adequacy in naming visual stimuli. First, the subject is prompted to practice by naming different colours, thereafter different shapes and thereafter different combinations of the two, with colour always being the first to be named. The test then commences with naming 40 colours presented in uniform shapes as quickly as possible. The stimuli are presented in random order on a plate. Thereafter, the same number of different black shapes is to be named. The same procedure is thereafter repeated with randomly presented combinations of the two stimuli, which the subject is asked to name as quickly as possible. The same procedure may be repeated with letters, numbers, animals, and objects. The total naming time of each of the subtests and the total number of erroneous naming of the stimuli, is assessed and is presented in a graph.

However, the speech pause time duration is not determined in '306. Moreover, no measure for separate components of brain function, like a component for processing for identification of stimuli, or a component for memory retrieval are provided in '306. For instance in naming tasks of large number of stimuli (e.g. consecutive naming of about 40 different shapes and colors and their combinations) it is not reliably determinable which shape or color or their combination is identified upon visual stimulation. The total time needed for the naming tasks comprises a mixture of many brain activity components, which are not individually identifiable from the total naming time. It is for instance not derivable which specific stimuli of a large number of stimuli presented during a naming task is processed during which portion of the naming time. This is primary due to the brain's ability of parallel processing and the phenomenon of “chunking”, i.e. the loading in memory of several stimuli in a sequence which is performed ahead of pronunciation, and which is a well-known phenomenon in reading. Moreover, the naming time is only provided as an average measure from a longer time of sequentially naming a large number of stimuli provided simultaneously.

Hence, an improved method of determining brain function testing would be advantageous, allowing for more detailed analysis of separate components of brain function. Such an analysis would be advantageous for diagnostic support of disorders related to the brain of a subject.

In addition, various methods and devices are known to test mental ability. For instance in U.S. Pat. No. 5,911,581 ('581) a method and apparatus for measuring and analyzing mental ability are disclosed. One aspect of '581 describes the assessment of reaction time by using a subliminal awareness threshold test whereby the outline of a stimulus (i.e. unfilled) is to be timely separated from a filled stimulus. The presentation time of the outlined stimulus begins at a subliminal threshold, and the presentation time is thereafter successively increased, until a subject can decide if the outlined stimulus came before the filled stimulus. After three correct responses by a subject, the presentation time of the outlined stimulus is set for further use. However, '581 is not based on speech analysis, which would be more advantageous, as voice based testing of mental abilities would be easier to perform. In addition, '581 is silent about diagnosis of disorders related to the brain, including neuropsychiatric disorders, or diagnostic support thereof.

Furthermore, United States patent application 2003/181793 ('793), and U.S. Pat. Nos. 5,230,629 ('529), and 4,770,636 ('636) disclose methods and devices for assessing memory by retrieval speed. In one embodiment of the disclosures, a subject is presented with a list of items and asked to correctly name the items in the correct order, thereby assessing the serial-processing memory and the retrieval speed of memory of that subject. Retrieval speed is measured by digital or computerized time pieces, but no voice recording is performed. In another embodiment of the art a speed monitor (Cognometer) is presented. This apparatus measures the difference between the time needed to copy arithmetic data and the time used to carry out an arithmetic computation on such numbers, and enter the results. The latency or time to copy or to subtract or add a number is measured from the onset of the number on the screen to the entry of the first digit of the two-digit response number. However, the disclosures '793, '529 and '636 do not mention any use for diagnosis of neuropsychiatric disorders, and are cumbersome, and not based on speech analysis.

The known methods and apparatuses may not be cultural- and educational independent. Tests performed by the known methods and apparatuses may thus be influenced by cultural- and educational background of the patients undergoing diagnosis or testing disorders related to the brain, including neuropsychiatric disorders.

Hence, there is a need for universal, cultural- and educational independent diagnosis or testing of disorders related to the brain, including neuropsychiatric disorders.

Adjustment of medication of disorders related to the brain, including neuropsychiatric disorders, based on speech analysis is not disclosed in any of the above referenced documents. However, there is a need of providing an easy way of adjusting such medication to the specific needs of a patient. Hence, there is a need for being able to provide a reliable adjustment of medication dosage to the specific needs of the patient. In particular, it would be advantageous to be able to provide a reliable adjustment of medication dosage of substances for treatment of neuropsychiatric disorders. Such an adjustment may for instance be based on a desired diagnostic support tool facilitating diagnosis of the progress of such disorders and effect of medication thereon.

Moreover, there is a need for being able to provide a relative measure of relative contribution of specific brain activity components to the total brain activity.

Hence, an improved apparatus and method would be advantageous and in particular allowing for increased flexibility, cost-effectiveness, reliability, versatility, independence of and/or patient friendliness would be advantageous.

### SUMMARY OF THE INVENTION

Accordingly, embodiments of the present invention preferably seek to mitigate, alleviate or eliminate one or more deficiencies, disadvantages or issues in the art, such as the above-identified, singly or in any combination by providing an apparatus, a method, and a computer program according to the appended patent claims.

The computational processing time of a visual stimulus before a subjects articulates the stimulus, is herein defined as “pause time duration”, or articulation start pause time duration.

The present invention takes advantage of this pause time duration as a measurable parameter for facilitating diagnosis of brain related dysfunctions, such as neuropsychiatric dysfunctions.

According to a first aspect of the invention, an apparatus is provided. The apparatus is adapted to provide diagnostic support of disorders related to the brain of a subject. The apparatus comprises a display for presenting stimuli to said subject, a microphone for registering a sound signal, and a control unit adapted to determine a recognition time for a stimulus as a first pause time component, in a first operational stage, determine a memory retrieval time for a stimulus as a second pause time component, in a second operational stage; and subsequently present on said display a pre-defined number of different stimuli individually and in an order, each stimuli being presented during said recognition time, and with a presentation pause of pre-defined length inbetween said individual presentations, and, after presenting of the last stimuli is finished, measure a pause time until articulation of said subject starts in said sound signal for naming the presented stimuli as quickly as possible and in said order, in a third operational stage. The apparatus may determine a presence, degree or risk of disorders related to the brain of the subject from said pause time until articulation of said subject starts. In diagnostic support a diagnosis may be made external to the apparatus and the apparatus provides for diagnostic support, e.g. by providing measurement based data of the diagnosed subject. Diagnostic support may facilitate diagnosing a progress of a disorder. This applies mutatis mutandis to other aspects of the invention, such as a method or computer program. Disorders related to the brain include amongst others neuropsychiatric disorders. The third operational stage may optionally be repeated a pre-defined number of times, e.g. to obtain a mean pause time value of a plurality of said measured pause times.

According to a second aspect of the invention, a method is provided for diagnosing disorders related to the brain of a subject, by using a voice signal. The method comprises: a first stage of determining a recognition time for a stimulus as a first pause time component, a second stage of determining a memory retrieval time for a stimulus as a second pause time component; and subsequently a pre-defined number of times a third stage of: presenting a pre-defined number of different stimuli individually and in an order, each stimuli being presented during said recognition time, and with a presentation pause of pre-defined length inbetween said individual presentations, and after presenting of the last stimuli is finished, measuring a pause time until articulation of said subject starts for naming the presented stimuli as quickly as possible and in said order. Disorders related to the brain include amongst others neuropsychiatric disorders. The method provides for determining, or diagnosing, a presence, degree or risk of said disorders related to the brain of the subject from said pause time until articulation of said subject starts.

According to a further aspect of the invention, a computer program for processing by a computer is provided. The computer program is for providing diagnostic support of disorders related to the brain, based on an analyzed voice signal. The computer program is devised for processing by a computer, the computer program comprising a plurality of code segments, including a first code segment for determining a recognition time for a stimulus as a first pause time component, a second code segment for determining a memory retrieval time for a stimulus as a second pause time component; and a third code segment for execution subsequently and a pre-defined number of times for presenting a pre-defined number of different stimuli individually and in an order, each stimuli being presented during said recognition time, and with a presentation pause of pre-defined length inbetween said individual presentations, and after presenting of the last stimuli is finished, measuring a pause time until articulation of said subject starts for naming the presented stimuli as quickly as possible and in said order.

In an embodiment, the computer program is embodied on a computer-readable medium.

Further embodiments of the invention are defined in the dependent claims, wherein features for the second and subsequent aspects of the invention are as for the first aspect mutatis mutandis.

Some embodiments of the invention provide for a relative measure of relative contribution of specific brain activity components to the total brain activity.

Some embodiments of the invention provide for an adjustment of medication dosage or type by an identification of brain activity components.

A brain activity component that can be measured is for instance the processing time for identification of a stimuli. Applicants have realized that this component is an important component for diagnostic support, for instance when diagnosing the progress of ADHD. Further, this component is influenced by specific medications that provide a drug effect on the cerebral processing.

Another brain activity component that can be measured is for instance the processing time for memory retrieval. Applicants have realized that this component is another important component, for instance when diagnosing ADHD. Further, this component is influenced by specific medications that provide a drug effect on the cerebral memory.

In this manner, an optimal adjustment of medication is provideable, e.g. by a combination of several drugs affecting specific components of brain activity.

Some embodiments of the invention provide for facilitating drug development. Specific drugs may be developed and the effect tested on subjects by the present method and apparatus. An improvement of one or more specific components of brain activity may be determined, as an effect of a specific medication.

In embodiments, an analysis of a sound file is performed, however, different than previously known. An objective provided by the present invention is to measure the relative contribution of several specific components of the pause time duration occurring before the vocal pronunciation of visual stimuli. Pause time is the time when the brain performs many lower and higher-order cognitive processes, before speech is articulated. The duration of this silent processing involves many different skills of the brain and is intrinsically associated with the speed of visual scanning, speed of stimulus discrimination and categorization, speed of encoding, retention and retrieval from working memory, and the formation speed of the phonological mechanisms subserving pronunciation of visual stimuli. These processes are universal and cultural- and educational independent. The overt articulation of stimuli is therefore the end-product of these foregoing mental processes and it has been shown in the art that articulation time is not associated with pause time duration. Therefore, being a much less sensitive measure of these processes, phonological output (articulation time) is therefore disregarded in the present invention.

Some embodiments further utilize a principle of serial-processing memory, by displaying four random colours or shapes, e.g. on a screen which are to be named correctly and in the correct order by a subject. At offset of the presentation of the stimuli, a voice recording assesses the pause time duration which occurs during the retrieval phase of the serially presented stimuli. The serial presentation of the stimuli may be repeated in order to provide a reliable measure of the mean pause duration time for each individual stimuli. The assessment of retrieval time in the present invention clearly differs from the art, in that memory retrieval herein is regarded and used as a component of the pause time duration which precedes the articulation of the stimulus. Thus, the pause time duration recorded during the retrieval and verbal naming of single stimuli is used by the present invention as a component of the pause time analysis in the sequential processing of discrete stimuli.

The disclosure of '306 clearly differs from the present invention on several important accounts.

First, although the stimulus of the present embodiments also involves different colours and shapes, the present invention is based on the assessment of voice recordings in naming the stimuli, and in particular the assessment of pause time duration occurring before the utterance of the stimuli. This is not made by the disclosure of '306.

Second, the present embodiments make use of the assessment of recognition and memory retrieval time and their respective contribution to pause time duration. This is not made by the disclosure of '306.

Thirdly, the presentation of the stimuli in the present embodiments is made up of discrete stimuli, contrary to the mentioned art. The two stimuli (colour and shape) are presented 0.1 second apart as default. This is not the case of the mentioned art, in which all stimuli are presented at once, and no allowance is made for manipulation of interstimulus duration. The deliberate separation of the stimulus by a short time interval of presentation time allows the present embodiments to manipulate the time distance between the stimuli, in order to probe the capacity of working memory in subjects. This is not possible by the disclosure of US2004/0058306.

Fourthly, the colour and shape combinations of the present embodiments can each be presented in a discrete sequence, i.e. one colour and one shape may be presented in random order, during which the voice recording assesses the pause time duration. Contrary to the mentioned art, the number of individual sequences of such pairwise presented stimuli can easily be increased or decreased.

Processing speed is a general and inherent aspect of brain function, and the ability of the brain to quickly process information is intricately linked to all mental operations. This function can be measured in many different ways and for all modalities. The wake brain is constantly exposed to visual information which needs to be processed. The speed, by which visual processing occurs, can be measured by for example asking a subject to pronounce the name of a visually presented stimulus. Thereby, processing speed is the summed time of all of the individual computational steps the brain uses to allocate attention and to visually recognize and categorize the stimulus, to encode and retrieve the stimulus from working memory, to perform phonemic processing, and finally to invoke the vocal apparatus to pronounce the stimulus. Any disturbances (morphological or neurochemical) of brain areas needed to perform all of these computations will inevitably lead to a reduction in the speed by which such information is processed and finally pronounced.

In addition, processing time, and in particular expressed by the duration of speech pauses between the articulations of stimuli, is a very sensitive measure of brain dysfunction in neurodegenerative disorders. This speech pause time duration is a predictor of learning and reading skills in children, and speech pause time duration decreases during stimulant medication. However, speech pause time duration does not provide a measure for separate components of brain function, like a component for processing for identification of stimuli, or a component for memory retrieval. For instance in naming tasks of a large number of stimuli (e.g. consecutive naming of 40 different shapes and colors and their combinations) it is not reliably determinable which shape or color is identified upon visual stimulation. Speech pause time, e.g. in naming tasks, comprises a mixture of many brain activity components, which are not individually identifiable from the speech pause time, due to the brain's ability of parallel processing and/or chunking of stimuli in working memory ahead of pronunciation. In the known art it is not derivable which specific stimuli of a large number of stimuli presented during a naming task is processed during which portion of the speech pause time. Moreover, often the speech pause time is only provided as an average measure from a longer time of sequentially naming a large number of stimuli provided simultaneously, in relation to total speech time.

It should be emphasized that the term “comprises/comprising” when used in this specification is taken to specify the presence of stated features, integers, steps or components but does not preclude the presence or addition of one or more other features, integers, steps, components or groups thereof.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a schematic illustration of a method of identifying a stimulus S recognition time—step one of an embodiment of a method;

FIG. 2 is a schematic illustration of identifying a memory retrieval time—step two of an embodiment of a method;

FIG. 3 is a schematic illustration of stimuli presentation and sound recording—step three of an embodiment of a method;

FIG. 4 is a schematic illustration of an embodiment of an apparatus for performing the method;

FIG. 5 is a schematic illustration of an embodiment of the method;

FIG. 6 is a schematic illustration of units of the apparatus;

FIG. 7 is schematic illustration of an embodiment of a computer program;

FIG. 8 is a graph illustrating the pause time duration (seconds) (P) in healthy, as well as dysfunctious non-medicated persons of different ages (A);

FIG. 9 is a schematic illustration of difference in pause time duration (seconds) between a drug-free and a medicated state of patients with Attention Deficit Hyperactivity Disorders (ADHD). Pause time duration is shown on the y-axis (P), while the bars show individual cases (I) along the x-axis. The results show a significant decrease in pause time duration after 1 hour of treatment with methylphenidate chloride;

FIG. 10 is a graph illustrating the variability in pause time duration before treatment (left) and after 1 hour of treatment with methylphenidate (right) in a group of ADHD. Pause time duration (seconds) (P) is shown on the y-axis and before (b) and during (a) treatment is shown on the x-axis. Circles denote the mean value, and bars denote the standard deviation.

FIG. 11 is a graph illustrating how an adolescent may deviate from normal. The graph shows an example of a 15-year old boy with ADHD. He has significantly delayed pause time duration for his age (A) before medication (horizontal arrow). Optimal medication with methylphenidate treatment showed a clear reduction in his pause time duration (seconds) (P) and a return to normal reference values (vertical arrow); and

FIG. 12 is a graph illustrating the difference in pause time duration (P, seconds) between children with mixed neurological disorders (H) and healthy ageing (C).

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

Disorders related to the brain include a range of neuropsychiatric disorders, some of which are of neurodevelopmental origin, such as autism spectrum disorders, attention deficit hyperactivity disorder (ADHD), learning disabilities, developmental delays and intellectual retardation. In addition to the many factors which contribute in complex ways to brain development, developmental disturbances may also include a host of comorbid conditions, some of which are associated with various neurological conditions in the brain. Some of the behavioural effects of such disorders may be associated with difficulties in the brain's capacity to process information and may include perceptual problems as well as memory difficulties.

The following description focuses on an embodiment of the present invention applicable to or facilitating diagnosis or providing diagnostic support of neuropsychiatric disorders and in particular to diagnosis or diagnostic support of ADHD. However, it will be appreciated that the invention is not limited to this application but may be applied to other disorders related to the brain including many other neuropsychiatric disorders including for example autism, and autism spectrum disorders; Aspergers syndrome; or brain damage induced disorders, such as Alzheimer's disease, Multiple sclerosis (MS) or any other sub-cortical white matter disease or demyelinating disease, HIV, malaria, cerebrovascular disease (VaD), encephalitis, traumatic brain injury (TBI), mild cognitive impairment (MCI), fronto-temporal dementia (FTD/FLD), dementia with Lewy body disease (LBD/DLB), Parkinson's disease (PD), and vascular dementia.

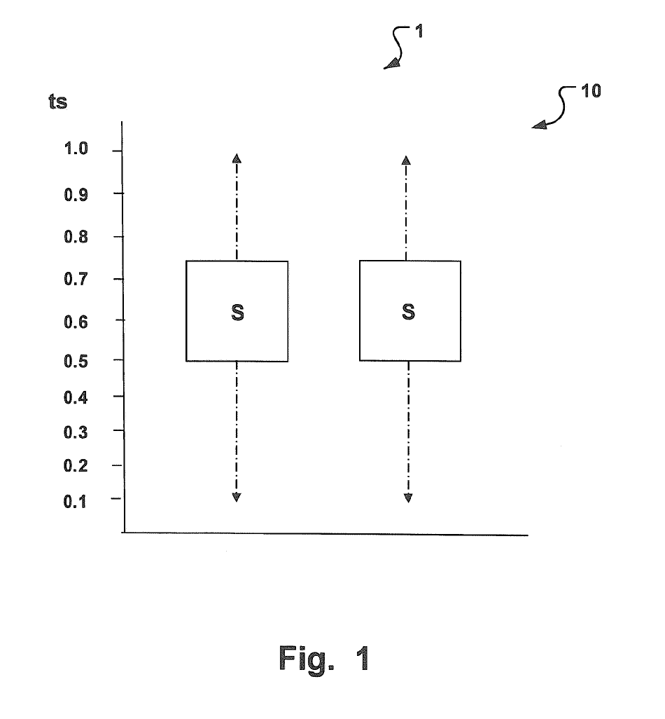

Embodiments of the invention are described in detail below with reference to FIGS. 1 to 12.

How to Perform the Measurement

The measurement is performed in three steps, depicted in FIGS. 1, 2, 3 and 4. These steps are performed in a fixed order. The default stimuli are four different colours and four different shapes, but the method is not limited to these stimuli. In other embodiments, other stimuli may be used alternatively or in addition, e.g. other visual stimuli, such as pictures; auditive stimuli, etc. The method is thus implemented in an education and culture-free manner, and wherein said measurement does not comprise questions related to knowledge of the subject.

Step 1: see FIG. 1. Assessment is made of recognition time for a stimulus—which is a part of the pause time measured in step 3).

As illustrated in the graph 10, identification of the stimulus recognition time is performed. A stimulus S is shown for a stimulation time ts (in parts of a second in FIG. 1). The stimulus S may be shown repeatedly with increasing time until a threshold time is reliably determined where the subject correctly identifies the presented stimulus. Adjustment is made of the lowest time level to correctly recognise the stimuli. For instance, the adjustment starts at the fastest level (0.1 seconds) and this time of stimulus presentation ts is increased successively until a stable recognition is achieved. Step 3 uses this recognition time threshold ts as a default time, as described below.

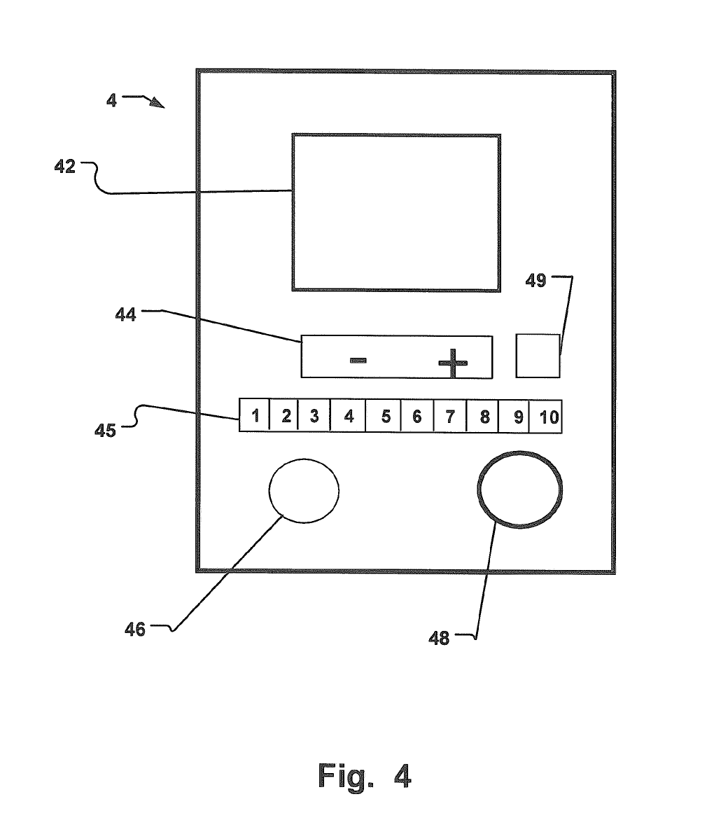

Thus, it is probed at which stimulus S presentation time the subject is able to correctly identify and name the stimulus. This is usually performed or supervised by an examiner, but may also be made software based by the subject alone using a suitable programmed apparatus. The stimulus presentation time can be changed at discrete levels ranging from the lowest level of 0.1 seconds up to the highest level of 1.0 second. The examiner establishes the lowest level for correct identification of the stimulus through the correct verbal response by the subject. When this presentation time is identified, the examiner (or subject) presses the OK-button 49 on the apparatus 4 (FIG. 4). This OK-signal is then automatically used as the presentation time of the stimulus in Step 3. In this manner a reliable and correct identification of the stimuli is ensured at a minimum presentation time. Thereby the component of recognition time for a stimulus—which is a part of the pause time—is reliably determinable.

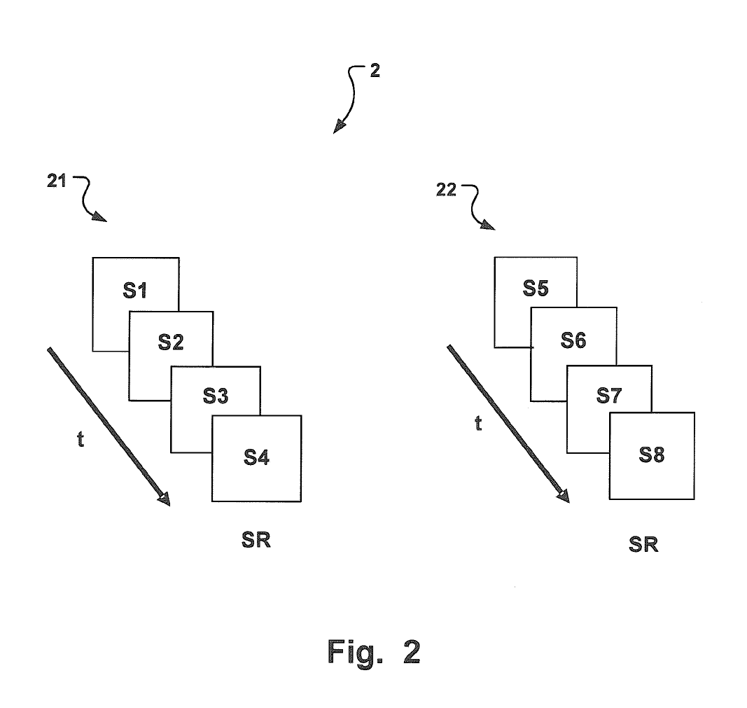

Step 2: see FIG. 2. Assessment is made of a memory retrieval time tr for a stimulus or probe of working memory—which is a part of the pause time measured in step 3.

A sequence or series 21 of a first number of stimuli, e.g. four colour stimuli, S1, S2, S3, S4, which are e.g. used as default stimuli, are presented on the screen 42 of the apparatus 4, see FIG. 4, over time t. Each first stimuli is visually presented at the set presentation time ts defined in Step 1. The presentation of the stimuli is made in successive order with pre-determined intervals, such as one second intervals, until all stimuli have been shown. The subject is instructed to name the series of first stimuli, in the embodiment the four colour stimuli S1-S4 correctly and in the correct order as soon as all four stimuli have been presented.

At offset of the last stimulus—here S4—sound recording SR of the anticipated speech is started and the pause time until speech actually starts after starting the SR is automatically analysed. Retrieval time tr for each individual stimulus is calculated as the mean pause time, i.e. the entire mean pause time divided by the number of presented stimuli—in the example for all four stimuli divided by four. The value for tr_mean is automatically stored, and is used by the apparatus 4 or method to calculate the amount of retrieval time contributing to the total pause time occurring in Step 3.

This procedure may be repeated for a second series 22 of stimuli S5, S6, S7, S8, e.g. four shapes that are different than the shapes of the first series 21.

This step 2 is a test of short-term memory and a measurement value therefor is provided by tr_mean. In more detail, by dividing the total pause time by the number of stimuli, the memory retrieval time tr_mean for one stimulus is determined.

In other embodiments, a different number of stimuli may be used.

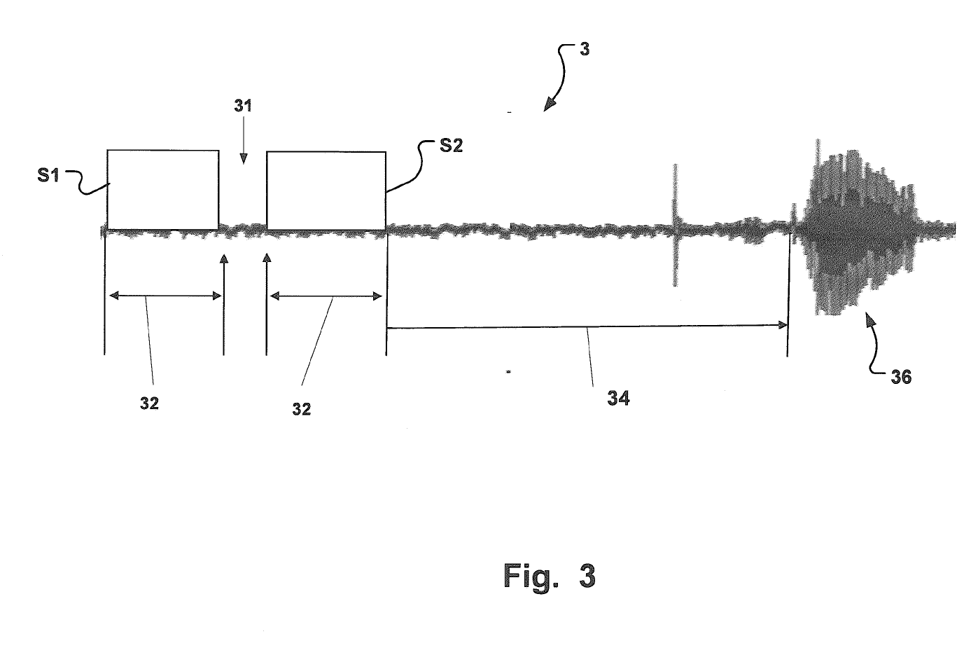

Step 3: see FIG. 3. Assessment is made of a pause time duration in pair wise presented stimuli.

Presentation of two visual stimuli. The stimuli are discrete and the presentation time 32 is separated by a pause time interval (presentation pause time 31). The minimum time (ts) to correctly identify the stimuli is determined by step 1. The stimulus presentation time ts set in Step 1 is used here. Presentation time 32 is preferably as large as ts, in order to ensure a reliable recognition of the two random stimuli S1 and S2 by the subject.

The presentation pause time 31 is adjustable and e.g. in the range of 0.1 seconds. This time interval of presentation pause 31 can be adjusted, but a default value of e.g. 0.1 seconds is used. The two stimuli 51, S2 (e.g. colour and shape) are separated by 0.1 second as a default value, thereby creating two discrete stimuli. This default value is a dynamic feature of the method and may be increased, e.g. for specifically probing a memory component by the present testing.

The individual stimuli 51, S2 as well as the order of the pair wise presentation of the stimuli S1, S2 is randomised (i.e. in the example sometimes a colour appears as the first stimulus S1, sometimes a shape appears first). The subject is required to always name the stimulus presentation in the correct order. Thus, the first presented stimulus S1 is to be named first. After the offset of the presentation of the second stimulus, the subject is required to name the stimuli S1 and S2 as quickly as possible.

The speech recording is automatically started at onset of the first stimulus, and the pause time duration 34 from the end of presenting the second stimulus S2 to the first vocal pronunciation (articulation 36) of the first stimulus by the subject, is analyzed.

The measured pause time 34 is here defined as the sum of the stimulus recognition time ts (lowest level in milliseconds, obtained in step 1), and the memory retrieval time tr_mean (The mean pause time for the 4 stimuli in step 2.)

The method automatically subtracts the stimulus presentation times and the interstimulus interval from the recorded sound signal. As sound recording is started in the embodiment before the end of presentation of stimulus S2, any recording delays or measurement errors are avoided. The amount of contribution that the memory retrieval time has contributed to the pause time duration, is analyzed. Further, the recognition time is analyzed. The total pause time duration may be longer than the added recognition time and retrieval time, due to other brain activity components.

Thus, the signal provides a measure of the total pause time duration, and components thereof.

The apparatus 4 is shown in an embodiment in FIG. 4. All above described three steps 1-3 may be performed by means of the apparatus 4.

The apparatus 4 has a screen 42, a button 44 for adjusting the presentation time of the stimuli, a microphone 46, a start button 48, and a time interval selector section or scale 45; e.g. 1=0.1 seconds, 2=0.2 seconds etc. The apparatus 4 is preferably a portable hand held apparatus. Optionally the apparatus may comprise communication means to communicate diagnostic results obtained in a suitable context. The buttons 44, 46, 48, 49 may be so-called soft-buttons, i.e. visually presented buttons on a touch sensitive display.

Step 1 is performed by adjusting the stimulus recognition time by means of scale 45, e.g. showing discrete intervals, for instance ranging between 0.1 to 1.0 seconds. The adjustment is performed by button 44 whereby the presentation time ts of the stimulus can be adjusted upwards (+) and downwards (−). When the minimum presentation time for a correct recognition and naming of the stimulus is identified, the OK-button 49 is pressed by the examiner/subject and this stimulus presentation time ts is thereby set as default value by the apparatus.

Step 2 (Retrieval time probing working memory) is performed in the manner presented above. The sound is registered by means of the microphone 46 and recorded in a memory of the apparatus 4 for further processing.

After Steps 1 and 2 have been performed, the subject may press the START-button 48. At this occasion, the pairwise stimuli are presented on the screen according to Step 3. Two stimuli 51, S2 are shown on the screen 42 and turned on and off in successive order, separated by the presentation pause time, e.g. 0.1 sec as the default value. The duration of each stimulus presentation ts is defined in step 1.

The onset of sound recording starts at the onset of the first stimulus 51 and stops after the pronunciation of the second stimulus has occurred, see FIG. 3. Thereafter, a pause is made, e.g. 1 second elapses (can be adjusted if necessary), until the next presentation of a stimulus pair occurs automatically or is started manually. That means, once the START-button 48 is pressed, the test may continue automatically with an adjustable time interval between presentations, until a plurality, e.g. fourty, randomly generated pairs of stimuli S1, S2 have been presented and a plurality of pause times is measured. Thus, the presentation sequences runs automatically a pre-defined number of times, e.g. 40 times as default. The apparatus automatically stops presentation of stimuli according to step 3, when this number is reached.

The individual sequences may also be presented separately and each separate sequence is thereby started by the examiner/subject by holding down the START-button, which automatically activates the individual sequence.

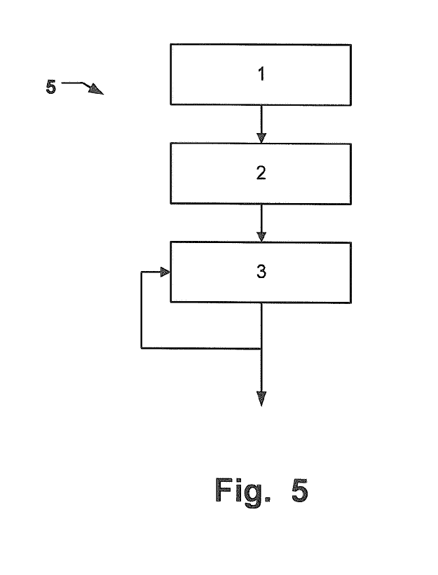

FIG. 5 is a schematic illustration of an embodiment of the method in a flow chart 5, comprising the above steps 1 to 3.

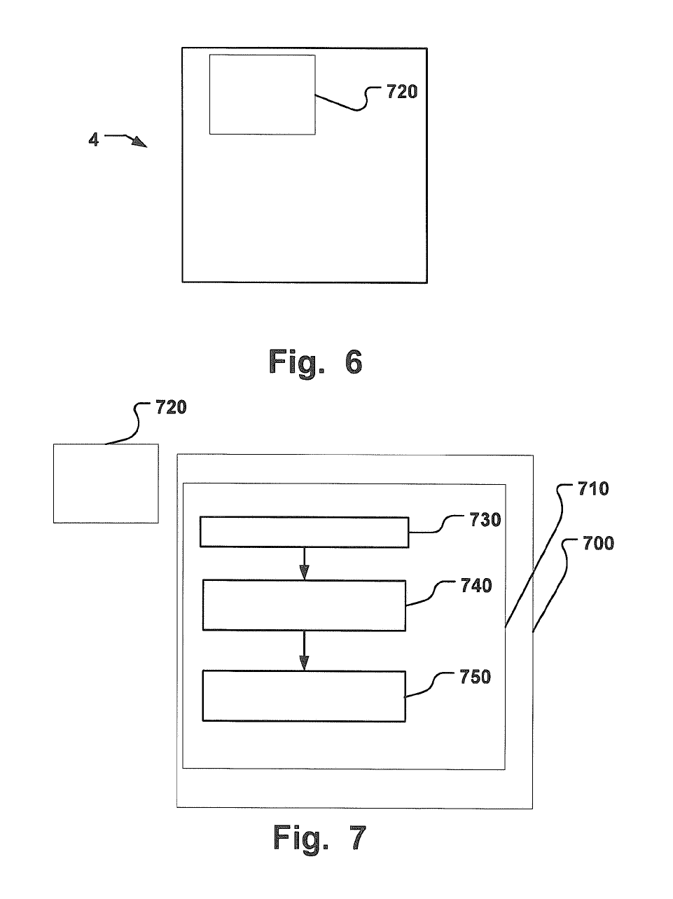

FIG. 6 is a schematic illustration of units of the apparatus comprising a control unit 720.

FIG. 7 is schematic illustration of an embodiment of a computer program 710, embodied on a computer readable medium 750, and comprising code segments 730, 740, 750.

In embodiments of the present invention, the assessment of a correct recognition of a stimulus differs from the previous art, such as disclosed in '581. In embodiments, a correct recognition is assessed by the successive increase (or decrease) of the presentation time of the particular stimuli itself, without reference to a predefined stimulus. The decision is made by the subject at which presentation time the stimulus is correctly and stably recognized and named, with reference to itself. Intrinsic to the present invention is the correct naming of the stimulus, not merely the correct visual discrimination of the stimulus. Contrary to the disclosure of '581, the present invention does not assess reaction time. Reaction time assessed by a correct identification of the order of stimulus presentation, as proposed in '581, occurs at a much quicker rate and involves a much earlier phase of visual processing. Instead, embodiments of the present invention assesses the summed end result of the whole series of processing events which take place before a subject is able to vocally name the stimulus correctly. This obtained presentation time for correct naming of the stimulus is set as a default parameter and is used in a series of the randomly ordered stimuli, which are presented at intervals, and after which the subject names the stimuli. A voice recording is performed after the last stimuli is presented to obtain the mean pause time duration during the naming of the individual stimulus to retrieve the memory retrieval time component.

There are various applications of the pause time measurement values and components determined therefrom, as provided according to above.

One application is an adjustment of medication to a desired dosage and/or type of medication.

Pause time may be shortened by the use of certain medication, as is demonstrated further below.

The above measurement may be performed before administering a medication, giving a first measurement value for relative comparison. Then, the medication is taken by the subject. A certain time after this administration, a new measurement is made, giving a second measurement value. The second measurement value relative the first measurement value gives a resulting effect of the medication. A shorter pause time is a clear indication of the effectiveness of the medication.

Different doses may thus be tested and the measurement value documents the effect of the medication. The present method and apparatus thus provide a tool for an assessment or evaluation of drug efficacy.

For instance, the dosage may be increased after a week of medication, e.g. if no effect on pause time is measured. The dosage may thus be titrated to an optimal level for a treatment window.

The present method and apparatus thus provide a tool for an adjustment of medication to individual patient needs.

Different components of the pause time, related to different brain activities may be identified individually. The effect of the medication on each individual brain activity component may thus be determined. For instance, the components are the above mentioned memory retrieval time and the stimulus recognition time.

The present method and apparatus thus provide a tool for clinical drug testing.

Further, a subject may be identified, who does not at all respond to medication (no change in pause time or components thereof). In this manner a further investigation may be initiated, as e.g. a neurological disorder prevents effectiveness of the medication.

A method, apparatus or computer program as described in unpublished application PCT/EP2008/064891 (diagnosis based on mean pause time in speech) of the same applicant as for the present application, which is incorporated herein by reference in its entirety for all purposes, may be used for diagnosing the effect of medication on neuropsychiatric diseases. This was not believed being possible at the time of filing PCT/EP2008/064891, but has now been demonstrated to be feasible against the prior view as described in PCT/EP2008/064891.

Table 1 shows a result of a patient study performed on 83 ADHD patients.

TABLE 1
Naming speed in ADHD (n = 83) who were assessed in a
medication-free state and after 1 hour of methylphenidate treatment

| | | Std. | Std | | | t- | Sign. |
|---|---|---|---|---|---|---|---|
| | Mean | dev. | Error | Minimum | Maximum | value | level |
| Total time | | | | | | | |
| 1 | 75.9 | 27.0 | 3.0 | 36.2 | 167.7 | | |
| 2 | 66.3 | 24.5 | 2.7 | 32.6 | 139.3 | 7.5 | 0.0001 |
| Pause time | | | | | | | |
| 1 | 37.0 | 20.8 | 2.3 | 8.8 | 110.7 | | |
| 2 | 29.3 | 18.4 | 2.0 | 5.5 | 85.6 | 6.2 | 0.0001 |
| Speech | | | | | | | |
| time | | | | | | | |
| 1 | 39.0 | 11.6 | 1.3 | 17.2 | 67.9 | | |
| 2 | 37.1 | 10.1 | 1.1 | 12.5 | 66.9 | 2.5 | 0.0132 |

1 = medication-free state,
2 = after 1 hour of medication, paired t-test.

TABLE 2

| | Before | After |
|---|---|---|
| Count | 19 | 19 |
| Mean | 13.0 | 6.9 |
| Std. Dev. | 12.8 | 4.1 |
| Maximum | 52.0 | 17.2 |
| Minimum | 2.1 | 1.6 |
| Range | 49.9 | 15.6 |

Table 2 shows the effect of medication on pause time variability in a group of children/adolescents with ADHD. In more detail, pause time variability during repeated measurements in ADHD before and after 1 hour of treatment with methylphenidate chloride. Clearly, it is demonstrated that the effect of the substance is measured by the embodied apparatus and/or method.

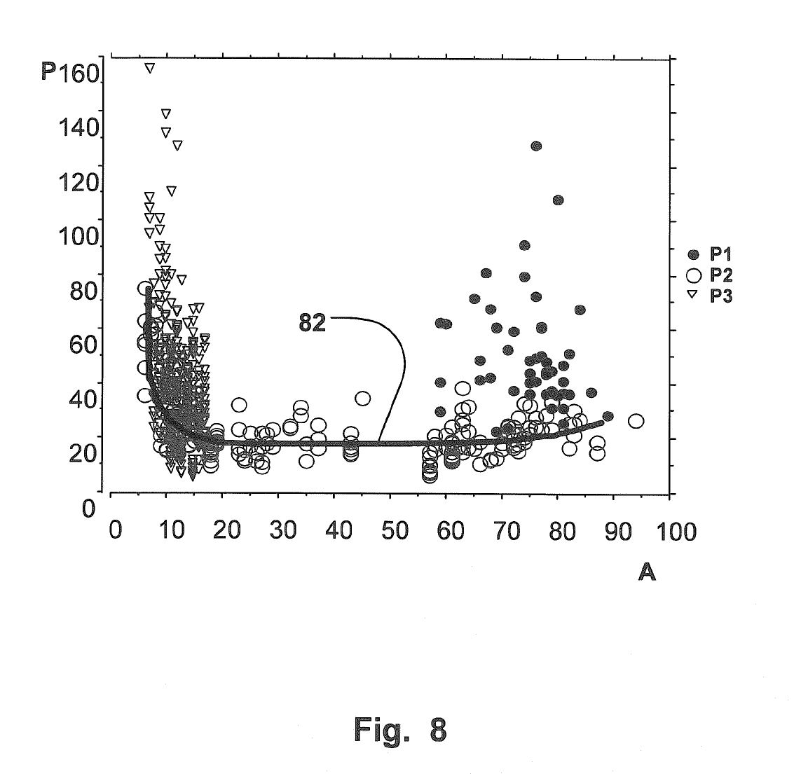

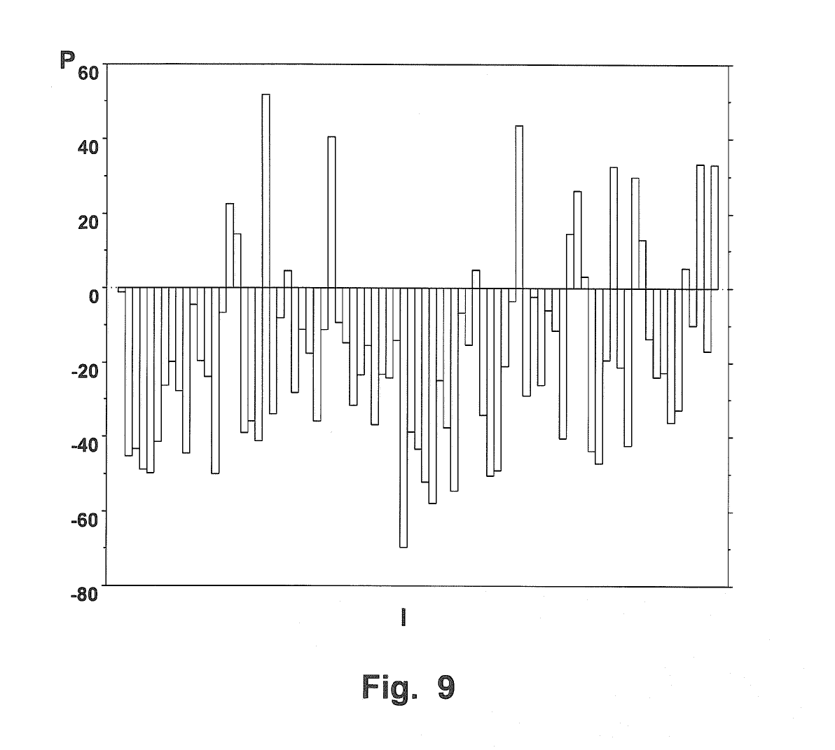

When referring to “pause time” with reference to Table 1, Table 2 and FIGS. 8-12, the term is directed to the speech pause component in a cognitive naming task, not to be confused with the above measured articulation start pause time duration. However, applicants envision that, proving the influence of medication on speech pause time, medication will also have an effect on the articulation start pause time duration after a finished stimuli presentation. By means of the afore described method and apparatus, the memory retrieval and stimulus recognition time are in addition determined.

As can be seen from Table 1, pause time is significantly affected (improved) by the administered medication. However, speech time (i.e. articulation time) is substantially not affected, see the significance levels in table 1, and FIGS. 8-12. The total time (articulation plus pause time) is also shorter upon medication intake. Total time foreshortening is predominantly based on shorter pause time.

In FIG. 8 the pause time duration P (in seconds) is plotted against age in three different subject groups, namely in healthy, as well as dysfunctious non-medicated persons of different ages (A). P3 (NPD) denotes Neuropsychiatric Disorders, P1 (Alzheimer) denotes Alzheimer's disease, and P2 (Norm) denotes normal controls. The solid line 82 shows the normal development of pause time duration P with increasing age A.

The graph shows that about half of the children with ADHD have pause time durations well above what is seen in healthy elderly, and in normal children. Furthermore, these children have similar pause time durations as those seen in patients with Alzheimer's disease.

FIG. 9 is a schematic illustration of the difference in pause time duration (seconds) between a drug-free and a medicated state of patients with Attention Deficit Hyperactivity Disorder (ADHD). Pause time duration is shown on the y-axis (P), while the bars show individual cases (I) along the x-axis. Hence, the FIG. 9 graph shows the difference in pause time duration P (in seconds) obtained at baseline (medication-free state) and after 1 hour of treatment with methylphenidate hydrochloride. As can be seen from this bar chart of individual cases, the vast majority of ADHD-children show a sizable reduction in the pause time duration during medication; in some cases the reduction is about 50-70 seconds. Thus the results show a significant decrease in pause time duration after 1 hour of treatment with methylphenidate chloride.

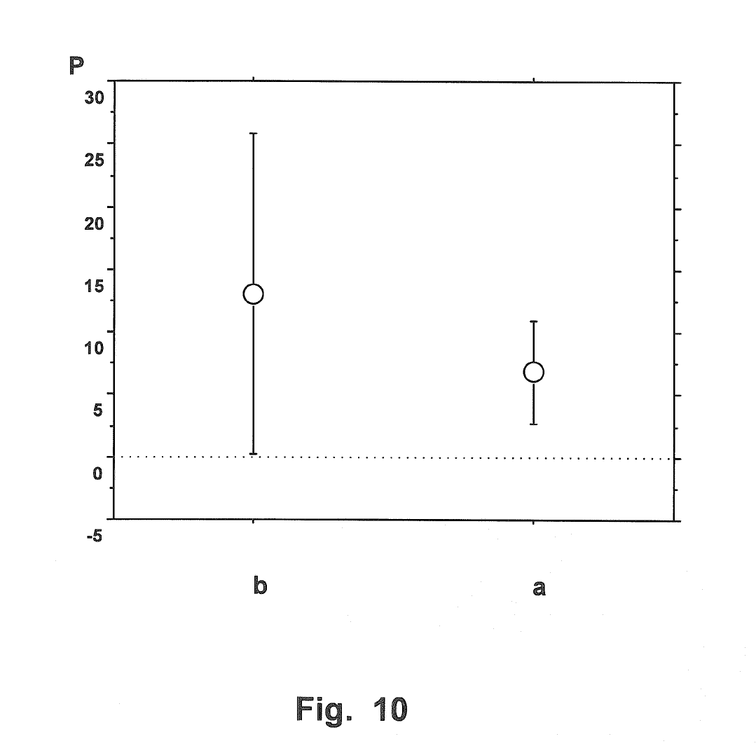

FIG. 10 is a graph illustrating the variability in pause time duration P (in seconds) before treatment (left bar) and after 1 hour of treatment with methylphenidate (right bar) in a group of ADHD. Pause time duration P (seconds) is shown on the y-axis, and before (b) and after (a) treatment is shown on the x-axis. Circles denote the mean value, and bars denote the standard deviation. Thus the FIG. 10 graph illustrates the difference in pause time variability between a drug-free and a medicated state of patients. The pause time variability obtained in a medication-free state (n=19) is compared with the variability obtained after 1 hour of methyphenidate hydrochloride treatment in the same patients. As can be seen, the pause time variability is clearly reduced, which is also shown in table 2. The pause time variability in FIG. 10 is here defined as the difference between the longest and the shortest pause time duration obtained in 3 consecutive testings which were performed at baseline and which were repeated again during medication. Thus, a reduction in variability indicates reduced pause time duration but also smaller differences between testings.

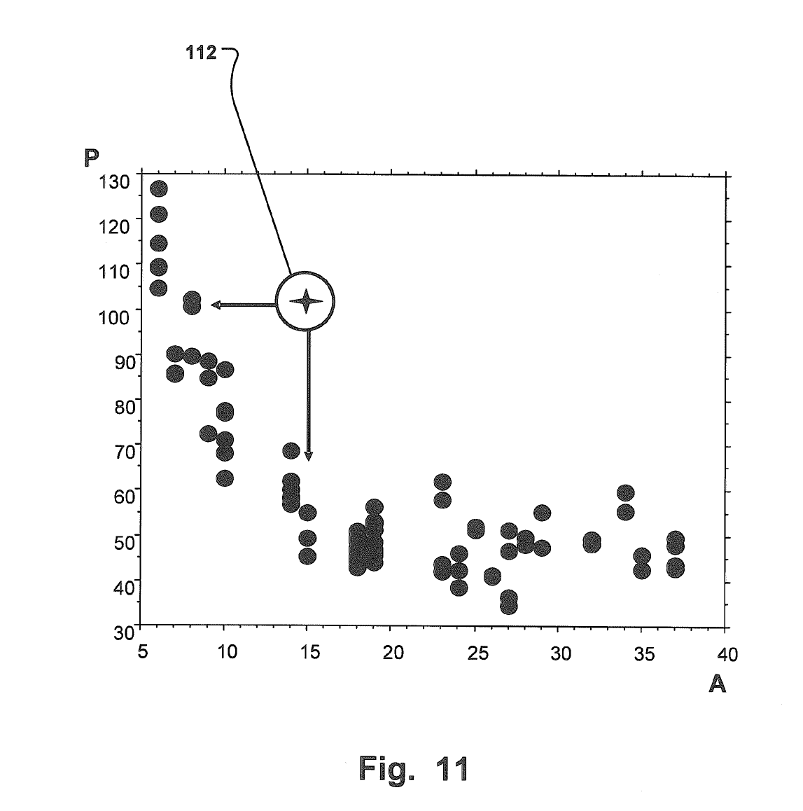

FIG. 11 is a graph illustrating how an adolescent may deviate from normal. The graph shows an example of a 15-year old boy 112 with ADHD. The case of the 15-year old boy is described to illustrate the degree to which pause time duration may be affected in response to treatment. He has a significantly delayed pause time duration for his age (A) before medication (horizontal arrow). Optimal medication with methylphenidate treatment showed a clear reduction in his pause time duration (seconds) (P) and a return to normal reference values (vertical arrow). As indicated by the arrows the treatment reduced pause time duration from about 100 seconds to about 60 seconds, i.e. an improvement by about 40%. This treatment improvement was made measureable by measuring the pause time P.

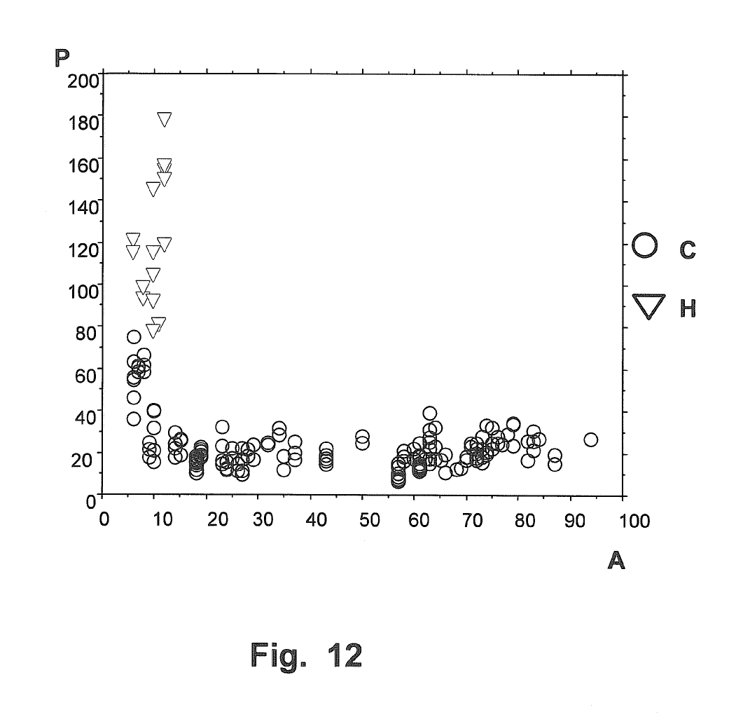

In FIG. 12 the pause time duration in a group of children with various neurological disorders (brain tumor, epilepsy etc) is shown in relation to normal reference values. The graph illustrates the difference in pause time duration (P, seconds) between the children with mixed neurological disorders (triangles) and healthy ageing (open circles). As can be seen, children with neurological disorders have a sizable increase in their pause time duration compared with normally expected values for their age. In certain embodiments, a diagnosis may thus be made by comparing measured pause times of an individual with an average pause time of a healthy population of the same age.

The present invention has been described above with reference to specific embodiments. However, other embodiments than the above described are equally possible within the scope of the invention. Different method steps than those described above, performing the method by hardware or software, may be provided within the scope of the invention. The different features and steps of the invention may be combined in other combinations than those described. The scope of the invention is only limited by the appended patent claims.

1. An apparatus adapted to support a diagnosis of disorders related to the brain of a subject, said apparatus comprising:
a display for presenting stimuli to said subject,
a microphone for registering a sound signal, and
a control unit adapted to
determine a recognition time for a stimulus as a first pause time component, in a first operational stage,
determine a memory retrieval time for a stimulus as a second pause time component, in a second operational stage,
present on said display a pre-defined number of different stimuli individually and in an order, each stimulus being presented during said recognition time, and with a presentation pause of pre-defined length in between said individual presentations, and
measure a pause time until articulation of said subject starts in said sound signal for naming the presented stimuli as quickly as possible and in said order, in a third operational stage.

2. The apparatus of claim 1, comprising a scale, showing discrete intervals ranging between 0.1 to 1.0 seconds for adjusting the stimulus recognition time by an input device such as a button.

3. The apparatus of claim 1, comprising a START-button adapted to start said presenting of stimuli in said third operational stage.

4. The apparatus of claim 2, wherein said stimuli are a pre-defined number of different visual and/or auditive stimuli.

5. The apparatus of claim 1, wherein said recognition time for a stimulus is determined by probing at which stimulus presentation time the subject is able to correctly identify and name the stimulus.

6. The apparatus of claim 5, wherein said stimulus presentation time is changeable at discrete levels from a lower level of 0.1 seconds towards a highest level of 1.0 second, wherein said recognition time is determined as the lowest level for correct identification of the stimulus through the correct verbal response by the subject.

7. The apparatus claim 1, wherein said control unit is adapted to determine said memory retrieval time for a stimulus by presenting a plurality of said stimuli, each at the presentation time determined in the first stage and in successive order with a time interval in between, and at offset of presenting the last stimulus being terminated, recording speech of said subject and analyzing pause time in said speech upon said subject naming the stimuli correctly and in the correct order, and calculating said memory retrieval time as the mean pause time for all stimuli divided by the number of stimuli.

8. The apparatus of claim 7, wherein said control unit is adapted to repeat said determining of said memory retrieval time according to claim 7 for a second series of different stimuli.

9. The apparatus of claim 1, wherein said pre-defined number of different stimuli in said third operational stage is two.

10. The apparatus of claim 1, wherein said presentation pause is at least 0.1 seconds.

11. The apparatus of claim 1, wherein said individual stimuli as well as the order of the presentation of the stimuli is randomized.

12. The apparatus of claim 1, comprising recording a sound signal at onset of the first stimulus, and the pause time duration to the first vocal pronunciation of the first stimulus, is determined from the recorded signal, and subtracting the stimulus presentation times and the interstimulus interval from the recorded sound signal, whereby a measure of the total pause time duration, and components thereof is provided.

13. The apparatus of claim 1, wherein said apparatus is adapted to apply said pause time and components thereof for diagnosis of neuropsychiatric disorders, including ADHD; autism spectrum disorders; or brain damage induced disorders of said subject.

14. The apparatus of claim 1, wherein said apparatus is adapted to apply said pause time and components thereof for an adjustment of medication dosage or medication type to individual patient needs of said subject.

15. The apparatus of claim 1, wherein said apparatus is adapted to apply said pause time and components thereof to identify subjects that do not respond to medication when no change in pause time occurs upon administration of said medication.

16. The apparatus of claim 1, wherein said apparatus is adapted to apply said pause time and components thereof for clinical drug testing and for evaluating and effect of said drug onto a neuropsychiatric disorder, including ADHD; autism spectrum disorders; or brain damage induced disorder of said subject.

17. A method for supporting diagnosing of disorders related to the brain of a subject, by using a voice signal, said method comprising:
a first stage of determining a recognition time for a stimulus as a first pause time component,
a second stage of determining a memory retrieval time for a stimulus as a second pause time component; and subsequently
a pre-defined number of times a third stage of:
presenting a pre-defined number of different stimuli individually and in an order, each stimulus being presented during said recognition time, and with a presentation pause of pre-defined length in between said individual presentations, and
after presenting of the last stimulus is finished, measuring a pause time until articulation of said subject starts for naming the presented stimuli as quickly as possible and in said order, wherein said pause time is a basis for diagnosing said disorders related to the brain of said subject.

18. The method of claim 17, wherein said stimuli are a pre-defined number of different visual and/or auditive stimuli.

19. The method of claim 17, wherein said recognition time for a stimulus is determined by probing at which stimulus presentation time the subject is able to correctly identify and name the stimulus.

20. The method of claim 19, wherein said stimulus presentation time is changed at discrete levels from a lower level of 0.1 seconds towards a highest level of 1.0 second, wherein said recognition time is determined as the lowest level for correct identification of the stimulus through the correct verbal response by the subject.

21. The method of claim 17, wherein said memory retrieval time for a stimulus is determined by presenting a plurality of said stimuli, each at the presentation time determined in the first stage and in successive order with a time interval in between, and at offset of presenting the last stimulus being terminated, recording speech of said subject and analyzing pause time in said speech upon said subject naming the stimuli correctly and in the correct order, and calculating said memory retrieval time as the mean pause time for all stimuli divided by the number of stimuli.

22. The method of claim 21, comprising repeating said procedure of claim 6 for a second series of different stimuli.

23. The method of claim 17, wherein said pre-defined number of different stimuli in said third stage is two.

24. The method of claim 17, wherein said presentation pause is at least 0.1 seconds.

25. The method of claim 17, wherein said individual stimuli as well as the order of the presentation of the stimuli is randomized.

26. The method of claim 17, comprising recording a sound signal at onset of the first stimulus, and the pause time duration to the first vocal pronunciation of the first stimulus, is determined from the recorded signal, and subtracting the stimulus presentation times and the interstimulus interval from the recorded sound signal, whereby a measure of the total pause time duration, and components thereof is provided.

27. (canceled)

28. The method of claim 17, wherein said pause time and components thereof are applied for supporting diagnosis of neuropsychiatric disorders, including ADHD; autism spectrum disorders; or brain damage induced disorders.

29. The method of claim 17, wherein said pause time and components thereof are applied for an adjustment of medication dosage or medication type to individual patient needs.

30. The method of claim 17, wherein said pause time and components thereof are applied to identify subjects that do not respond to medication when no change in pause time occurs upon administration of said medication.

31. The method of claim 17, wherein said pause time and components thereof are applied for clinical drug testing and for evaluating and effect of said drug onto a neuropsychiatric disorder, including ADHD; autism spectrum disorders; or brain damage induced disorder of said subject.

32. A computer program for supporting diagnosing disorders related to the brain, by using a voice signal, said computer program being devised for processing by a computer, the computer program comprising a plurality of code segments, including:
a first code segment for determining a recognition time for a stimulus as a first pause time component,
a second code segment for determining a memory retrieval time for a stimulus as a second pause time component; and a third code segment for execution subsequently and a pre-defined number of times for presenting a pre-defined number of different stimuli individually and in an order, each stimuli being presented during said recognition time, and with a presentation pause of pre-defined length in between said individual presentations, and after presenting of the last stimulus is finished, measuring a pause time until articulation of said subject starts for naming the presented stimuli as quickly as possible and in said order.

33. The computer program of claim 32, embodied on a computer-readable medium.

34. (canceled)

35. A method of internally controlling the apparatus of claim 1, said method comprising:
presenting stimuli to said subject on said display,
registering a sound signal from said subject by said microphone,
said control unit determining a recognition time for a stimulus as a first pause time component from a first registered sound signal, in a first operational stage,
said control unit determining a memory retrieval time for a stimulus as a second pause time component, in a second operational stage from a second registered sound signal; and
said control unit subsequently controlling a presentation of a pre-defined number of different stimuli individually and in an order on said display, each stimuli being presented during said recognition time, and with a presentation pause of pre-defined length in between said individual presentations, and, after presenting of the last stimuli is finished, said control unit measuring or determining a pause time from a third registered sound signal until articulation of said subject starts in said third sound signal for naming the presented stimuli as quickly as possible and in said order, in a third operational stage.
